(19) United States
(12) Patent Application Publication
(10) Pub. No.: US 2007/0092470 A1
(43) Pub. Date: Apr. 26, 2007

(54) Liquid, PEG-free, cold-processable oil-in-water emulsifiers

(75) Inventors: Petra Allef, Bonn (DE); Peter Hameyer, Essen (DE); Jurgen Meyer, Munster (DE)

Correspondence Address: Leopold Presser;Scully, Scott, Murphy & Presser, 400 Garden City Plaza, Garden City, NY, 11530, US

(73) Assignee: Goldschmidt GmbH, Essen (DE)

(21) Appl. No.: 11/585,540

(22) Filed: Oct. 24, 2006

(30) Foreign Application Priority Data
Oct. 26, 2005 (DE) 10 2005 051 222.4

(51) Int. Cl. A61K 8/37; A61K 8/73

(52) U.S. Cl. 424/70.13; 424/70.31

(57) ABSTRACT

The invention provides liquid, cold-processable oil-in-water emulsifier systems comprising a liquid, PEG-free emulsifier base consisting of A) one or more polyol partial esters, Ba) one or more acid partial esters carrying neutralizable acid functions, or Bb) one or more acid partial esters carrying at least partially neutralized acid functions, and C) optionally, polar liquid solubility promoters.

### FIELD OF THE INVENTION

The present invention relates to liquid, polyethylene glycol (PEG)-free, cold-processable oil-in-water emulsifier combinations comprising liquid emulsifiers based on polyol partial esters, preferably sorbitan or polyglycerol partial esters, and at least partially neutralized acid partial esters, preferably fruit acid partial esters, and optionally the amount of polar solubility promoters necessary for achieving single-phase systems, preferably water. The instant invention also relates to the use of these emulsifiers for the preparation of cosmetic, pharmaceutical and technical formulations, in particular of emulsions, and also to a method of producing and stabilizing oil-in-water emulsions using the emulsifier combinations according to the invention.

### BACKGROUND OF THE INVENTION

For ecological reasons, there is considerable interest in oil-in-water emulsifiers which are based on native raw materials both from the manufacturers and also from the consumers of emulsion preparations. These are preferably emulsifiers which comprise no polyethylene glycol-containing radicals (“PEG-free” emulsifiers). For this reason, partial esters of polyalcohols, such as glycerol, polyglycerol, sorbitol or methyl glycoside and fatty acids, such as lauric acid, oleic acid or isostearic acid, are used widely. Many of these emulsifiers are cloudy and have a tendency to separate.

A disadvantage of all of the emulsifiers mentioned is the fact that they are of pasty to solid consistency and therefore have to be melted prior to use. In times when process sequences have to be optimized and energy costs have to be restricted, this means a considerable competitive disadvantage compared with emulsifiers which are liquid at room temperature and can thus be processed directly without prior heating.

EP-A-1 250 916 describes a mixture of polyglycerol partial esters and sorbitan partial esters which is cold-processable. In particular, EP '916 discloses polyglycerol partial esters of C₈ to C₁₈, preferably C₁₂, fatty acid with polyglycerol-5 to polyglycerol-15, preferably polyglycerol-10, and sorbitan partial esters of C₈ to C₁₈, preferably C₁₂, fatty acid and sorbitol. However, these emulsifiers cannot be used universally and often do not form storable-stable emulsions.

JP-A-60-262827 describes a liquid mixture for the emulsification of silicone oils consisting of C₁₂₋₁₈-sorbitan partial esters, C₁₂₋₁₈-polyglycerol partial esters and silicone oils.

EP-B-0 853 494 describes the use of mixtures of an oil-in-water emulsifier (alkyl polyglycosides and/or fatty acid N-alkylpolyhydroxyalkylamides) and a water-in-oil emulsifier (polyolpolyhydroxystearates) as liquid, readily pumpable oil-in-water emulsifiers. In order to obtain readily pumpable systems, the emulsifier is diluted with polyols, preferably glycerol.

The same hydrophilic emulsifier system is described in DE-A-103 34 225 and DE-A-103 46 515 in combination with alkyl acylglutamates for preparing nanoemulsions.

DE-A-198 37 841 describes the use of aqueous dispersions of emulsifier and wax bodies for the cold preparation of emulsions. However, the preparation of the dispersions takes place at the melting temperature of the wax. No statements are made about the storage stability of the dispersions. As a rule, these dispersions always have to be freshly prepared, which at least minimizes the time advantage overall.

The cold-processable PEG-free emulsifier systems described in the prior art generally lead to moderate emulsion stabilization, which, depending on the system to be emulsified, leads to limitations, which is an obstacle to these systems being used universally.

In view of the above, there is a need for providing new and improved PEG-free oil-in-water emulsifiers that are liquid at room temperature and thus can be used for the cold preparation of emulsions with very good emulsion stability, giving rise to considerably broader possible uses of these systems.

### SUMMARY OF THE INVENTION

Surprisingly, it has now been found that liquid, PEG-free polyol partial esters, which are either oil-in-water emulsifiers with an inadequate emulsifier activity on their own, or are water-in-oil emulsifiers can be converted into high-performance, cold-processable oil-in-water emulsifiers by adding small amounts of emulsifiers which carry at least partially neutralized acid function.

The performance of these new types of cold-processable PEG-free emulsifiers is evident from their broad applicability for emulsifying highly diverse cosmetic oils, and in the low emulsifier concentrations (e.g., 1 to 3% by weight, based on the total emulsion) which are required for forming stable emulsions.

Here, the required amounts of the added emulsifiers with at least partially neutralized acid functions can optionally be incorporated into the liquid emulsifier base by adding polar solubility promoters (in the simplest case water) such that a single-phase, preferably clear system is formed. A mixture consisting of 70 to 90% by weight of sorbitan ester, 8 to 20% by weight of hydrophilic polyglycerol ester and 2 to 10% by weight of neutralized citric acid partial ester has proven to be particularly suitable. In accordance with the present invention, the best results are obtained if the hydrophobic radical used in all three emulsifier components is lauric acid.

The present invention thus provides liquid, cold-processable oil-in-water emulsifier systems comprising a liquid, PEG-free emulsifier base consisting of
A) one or more polyol partial esters;
Ba) one or more acid partial esters carrying neutralizable acid functions, or
Bb) one or more acid partial esters carrying at least partially neutralized acid functions; and
C) optionally, polar liquid solubility promoters.

### DETAILED DESCRIPTION OF THE INVENTION

As stated above, the present invention provides liquid, cold-processable oil-in-water emulsifier systems comprising a liquid, PEG-free emulsifier base consisting of one or more polyol partial esters; and one or more acid partial esters carrying neutralizable acid functions, or one or more acid partial esters carrying at least partially neutralized acid functions. Polar liquid solubility promoters may optionally be present in some embodiments of the present invention.

In accordance with the present invention, the liquid base component (A) consisting of polyol partial esters comprises one or more emulsifiers, preferably selected from at least one of the following groups:
A1) sorbitan or sorbitol partial esters, preferably preparable by esterification of aliphatic, linear or branched, optionally unsaturated and/or hydroxy-functionalized carboxylic acids with a chain length of from 6 to 22 carbon atoms with sorbitol,
A2) glycerol and polyglycerol partial esters, preferably preparable by esterifying aliphatic, linear or branched, optionally unsaturated and/or hydroxy-functionalized carboxylic acids with a chain length of from 6 to 22 carbon atoms with glycerol, polyglycerols or mixtures of the two,
A3) carbohydrate esters, preferably glycoside or sucrose esters, preferably preparable by esterification of aliphatic, linear or branched, optionally unsaturated and/or hydroxyl-functionalized carboxylic acids with a chain length of from 6 to 22 carbon atoms with mono- or polysaccharides,
A4) (alkyl poly)glycosides, preferably preparable by reacting aliphatic, linear or branched, optionally unsaturated and/or hydroxy-functionalized alcohols or alkyl halides with a chain length from 6 to 22 carbon atoms with mono- or polysaccharides.

Typically, the majority of the liquid polyol partial ester component (A) consists of sorbitan esters, admixed to which are preferably hydrophilic polyglycerol esters in an amount from 0 to 30% by weight, preferably 3 to 25% by weight, particularly preferably 8 to 20% by weight.

Preference is given in the present invention to a combination based on sorbitan and polyglycerol partial esters which comprise fatty acid radicals with a chain length from 10 to 16 carbon atoms as hydrophobic components.

More preference is given in the present invention to using a combination of sorbitan laurates and polyglycerol laurates.

The base component (B)—carrying a neutralizable or at least partially neutralized acid function—can comprise one or more emulsifiers, preferably selected from at least one of the following groups:
B1) Di- or polycarboxylates optionally containing hydroxyl groups, sulfated, sulfonated or phosphated carboxylates, malonates, malates, succinates, sulfosuccinates, citrates, tartrates in which the acid groups have been partially esterified with aliphatic or aromatic, linear or branched, optionally unsaturated and/or hydroxy-functionalized alcohols with a chain length of from 6 to 22 carbon atoms,
B2) Di- or polycarboxylates optionally containing hydroxyl groups, sulfated or sulfonated or phosphated carboxylates, malonates, malates, succinates, sulfosuccinates, citrates, tartrates in which the acid groups have been partially esterified with polyols, polyol partial esters, preferably of glycerol, polyglycerol and/or sorbitol with aliphatic or aromatic, linear or branched, optionally unsaturated and/or hydroxy-functionalized carboxylic acids with a chain length of from 6 to 22 carbon atoms,
B3) polyols, preferably glycerol, polyglycerol and sorbitol, which are partially esterified with aliphatic or aromatic, linear or branched, optionally unsaturated and/or hydroxy-functionalized mono-, di- or polycarboxylic acids with a chain length of from 2 to 22 carbon atoms, with the proviso that there are free, neutralizable acid groups in the molecule,
B4) hydroxy-functional, mono-, di or polycarboxylic acids whose hydroxyl groups have been reacted at least partially with aliphatic, linear or branched, optionally unsaturated and/or hydroxy-functionalized carboxylic acids with a chain length of from 6 to 22 carbon atoms,
B5) N-acylamino acids, such as sarcosinates, glutamates, aspartates, comprising an aliphatic or aromatic, linear or branched, optionally unsaturated and/or hydroxy-functionalized radical with a chain length of from 6 to 22 carbon atoms,
B6) carboxylates, sulfates, sulfonates or phosphates, comprising an aliphatic or aromatic, linear or branched, optionally unsaturated and/or hydroxy functionalized radical with a chain length of from 6 to 22 carbon atoms.

The emulsifiers of type (B) are present in the emulsifier formulation preferably in at least partially neutralized form (Bb). The emulsifiers of type (B) are advantageously used already as (partially) neutralized components. If desired, the neutralization step can, however, also take place in a suitable later processing step, in which case bases are preferably used for the neutralization that lead to anion-active emulsifiers with monovalent or divalent cationic counterions. Particularly preferred counterions employed in the present invention are sodium and potassium.

The emulsifiers of type (B) are used in amounts of at most 20% by weight, preferably ≦10% by weight, particularly preferably ≦5% by weight, based on the liquid emulsifier base consisting of polyol partial esters.

Preference is given in the present invention to using neutralized citric acid partial esters whose hydrophobic radicals each contain 10 to 16 carbon atoms.

More preference is given in the present invention to using the partial esters of citric acid and lauryl alcohol or of citric acid and glycerol mono- or dilaurates.

Liquid polar solubility promoters used for producing single-phase, clearest possible cold-processable oil-in-water emulsifier systems according to the invention include water, glycols, polyalkylene glycols, glycerol, polyglycerols, and alcohols in a total amount of at most 20% by weight, preferably at most 10% by weight, of the total emulsifier system. Preferably, water is used as the liquid polar solubility promoter. The amount of required polar solubility promoter is essentially dependent on the type of emulsifier and emulsifier ratio and can be ascertained easily through manual experiments. As a rule, amounts of from 2 to 10% by weight of solubility promoters suffice.

The invention further provides the use of the inventive oil-in-water emulsifier systems for the preparation of cosmetic, dermatological or pharmaceutical preparations. These preparations are preferably emulsions, which may optionally also comprise dispersed solids.

The invention further provides the use of the inventive oil-in-water emulsifier systems for the preparation of care and cleaning compositions for domestic use and industry, in particular for hard surfaces, leather or textiles. These preparations are preferably emulsions, which may optionally also comprise dispersed solids.

It has been found that exceptionally stabilized emulsions can be obtained during cold preparation both when the emulsifier combination according to the invention is added directly to the oil phase or water phase of the emulsion, and when the nonionic emulsifier component (A) is used in the oil phase, and the partially neutralized emulsifier component (B) is used separately therefrom directly in the water phase.

The invention further provides a method for the cold preparation of oil-in-water emulsions, wherein, using an emulsifier combination of
A) a PEG-free liquid emulsifier base consisting of one or more polyol partial esters (A),
B) acid partial esters (Ba) carrying one or more neutralizable acid functions,
C) optionally, polar solubility promoters, and
D) an amount of a base sufficient for the at least partial neutralization of the acid groups (Ba),
   oil phase and water phase of the overall emulsion are homogenized in the customary manner, optionally with co-use of auxiliaries and additives.

The invention also provides a method for the cold preparation of oil-in-water emulsions wherein, using an emulsifier combination according to the present invention of
A) a PEG-free liquid emulsifier base consisting of one or more polyol partial esters (A),
B) one or more emulsifiers which carry an at least partially neutralized acid function (Bb), and
C) optionally, polar solubility promoters,
   oil phase and water phase are homogenized in the customary manner, optionally with co-use of auxiliaries and additives.

The invention also further provides a method for the cold preparation of oil-in-water emulsions using an emulsifier combination according to the invention, wherein the nonionic emulsifier component (A) is added to the oil phase and the partially neutralized emulsifier component (Bb) is added, separately, to the water phase, and the two phases are homogenized in the usual way, optionally with co-use of auxiliaries and additives.

It has also been found that it is possible to initially introduce emulsifier component (B) together with, or separately from, emulsifier component (A) in a non-neutralized form in the water phase or oil phase and to only carry out a partial neutralization directly prior to the homogenization step.

The invention thus further provides a method for the cold preparation of oil-in-water emulsions, wherein the emulsifier component (B) is initially introduced in a non-neutralized form together with or separately from emulsifier component (A), optionally with co-use of auxiliaries and additives, in the water phase or oil phase, and the partial neutralization of emulsifier component (B) only takes place directly prior to the homogenization step carried out in the usual manner.

Cold preparation in the usual manner means that the emulsion can be produced at ambient temperatures without the otherwise required additional heating of the components.

For this, it is necessary that emulsifiers according to the invention are in the form of liquid, pumpable systems at these ambient temperatures.

Ambient temperatures are understood here as meaning temperatures in the range from about 10° to 50° C., but preferably Central European room temperatures in the range from about 15° to 35° C. Although heating is also likewise possible, it affords no advantages in terms of processing.

Preference is given to the use of the oil-in-water emulsifiers according to the invention for the cold preparation of cosmetic formulations for the care and cleansing of skin and hair.

These may, for example, be creams or lotions for skin care, products based on surfactants for the cleaning and care of skin and hair, sunscreen products, pigment-containing products from the field of decorative cosmetics (e.g., make up, products for lid/eyelash coloring), products for conditioning hair, nail care products or antiperspirant/deodorants.

In these formulations, the oil-in-water emulsifiers according to the invention can be used together with auxiliaries and additives known to the person skilled in the art as prior art, such as oils and waxes, cosurfactants and coemulsifiers, consistency regulators, thickeners, e.g., based on polymer, UV photoprotective filters, self-tanning agents, antioxidants, hydrotropes, deodorant and antiperspirant active ingredients, active ingredients, dyes, preservatives and perfumes.

Preferred active ingredients include tocopherol, tocopherol acetate, tocopherol palmitate, ascorbic acid, deoxyribonucleic acid, coenzyme Q10, retinol and retinyl derivatives, bisabolol, allantoin, phytantriol, panthenol, AHA acids, amino acids, hyaluronic acid, creatine (and creatine derivatives), guanidine (and guanidine derivatives), ceramides, phytosphingosine (and phytosphingosine derivatives), sphingosine (and sphingosine derivatives), pseudoceramides, essential oils, peptides, protein hydrolyzates, plant extracts and vitamin complexes.

The particularly preferred, cold-preparation oil-in-water emulsions may be stabilized by adding known polymer thickeners, such as, for example, polysaccharides, in particular xanthan gum, guar and guar derivatives, agar and agar derivates, alginates and tyloses, cellulose and cellulose derivatives, such as, for example, carboxymethylcellulose, hydroxyethylcellulose, hydroxymethylpropylcellulose, also alkyl-modified sugar derivatives, such as, for example, cetylhydroxylethylcellulose. Particular preference is given to using carbomers (crosslinked polyacrylates), which may also be alkyl-modified, also polyacrylamides or copolymers, which may be constructed, for example, from components such as acrylic acid, methacrylic acid, acrylamide or acrylamidopropanesulfonic acid, in addition also polyvinyl alcohol and polyvinylpyrrolidone.

It could not be deduced from the known prior art that by adding small amounts of neutralized fruit acid partial esters, it is possible to produce widely usable, cold-processable oil-in-water emulsifier from a weakly active to inactive oil-in-water emulsifier system.

The following examples demonstrate that an emulsifier such as sorbitan mono/di/triester (INCI: sorbitan laurate) whose emulsifying activity is known to be inadequate for producing stable oil-in-water emulsions can be converted, through combination with neutralized citric acid partial esters, into a cold-processable PEG-free emulsifier with good stabilization potential.

It is also demonstrated that an emulsifier mixture of sorbitan esters and polyglycerol partial esters which is not suitable for producing oil-in-water emulsions can likewise be converted into a highly effective, cold-processable PEG-free emulsifier through combination with neutralized citric acid partial esters.

The combination according to the invention of polyol partial esters in particular with citric acid partial esters whose good biodegradability is known from the food sector makes available an emulsifier mixture, which is also advantageous from ecological points of view, having a broad application diversity. Moreover, the use according to the invention for the cold preparation of emulsions ensures a resourceful approach as regards energy.

In this economically and ecologically extremely advantageous core aspect of the present invention of relevance in practice, its enormous advantages are clearly evident in the preparation and stabilization of cold-processable, PEG-free oil-in-water emulsions.

### EXAMPLES

The following example emulsions serve to illustrate the subject-matter of the invention in more detail without limiting it to these examples.

The concentration data in all examples are given as % by weight.

The emulsions were prepared such that oil phase and water phase were combined at room temperature. Homogenization was then carried out. The pH of the emulsions was adjusted to 5.5 to 6.

The stabilizers used were a customary combination of xanthan gum and carbomer, which were added in the form of an oily dispersion to the oil phase.

For the examples, formulations with lotion-like consistency were chosen.

In particular, the examples show how the use of a cold-processable emulsifier mixture which comprises amounts according to the invention of a partially neutralized emulsifier component (B) containing acid groups, allows oil-in-water emulsions with good to very good stability to be obtained.

The comparison examples show that stable emulsions are not obtained if the addition of the emulsifier component (B) according to the invention is dispensed with and only the liquid base component (A) is used.

These examples thus illustrate the technical teaching of this application, which shows how, through the combination according to the invention of two emulsifier components—which by themselves are not suitable for the cold preparation of emulsions—highly effective, versatile, cold-processable oil-in-water emulsifiers can be obtained.

In order to demonstrate the versatility of the emulsifier systems according to the invention, when choosing the oils used, recourse was mostly made to an emollient with high polarity (capryl/capric triglycerides) and an emollient with very low polarity (paraffinum perliquidum). In some cases, an emollient with moderate polarity (ethylhexyl palmitate) was also used.

When assessing the stability of the emulsions, unstable means that such systems exhibited water or oil separation during the storage time/storage conditions (3 months at room temperature and 45° C.; three freeze-thaw cycles between room temperature and −15° C.).

The term ‘very good stability’ is used in the present invention to denote that these emulsions displayed no instabilities of any type such as, for example, signs of phase separation, changes in viscosity or degree of dispersion.

For emulsions with good stability, the same is true with the limitation that according to the low-temperature test, a gradual decrease in the white color of the emulsions was observed, but in no case water or oil separation.

Description of the emulsifier systems used in the example formulations (the total percentage per emulsifier system adds up to 100 in each):

Emulsifier 1:

| | |
|---|---|
| Emulsifier component A: | 85% sorbitan laurate¹⁾ |
| | 8% polyglyceryl-4 laurate²⁾ |
| Emulsifier component B: | 3% potassium diglyceryl monolaurate citrate |
| Polar solubility | 4% water |
| promoter: | |

¹⁾TEGO ® SML (Degussa)
²⁾TEGO ® Care PL 4 (Degussa)

Comparison Emulsifier 1:
Exclusively emulsifier component A (to 100%, i.e., 91.4% sorbitan laurate and 8.6% polyglyceryl-4 laurate).
Potassium diglyceryl monolaurate citrate is the potassium salt of the diester of citric acid and glyceryl monolaurate.

Emulsifier 2:

| | |
|---|---|
| Emulsifier component A: | 85% sorbitan laurate¹⁾ |
| | 8% polyglyceryl-4 laurate²⁾ |
| Emulsifier component B: | 3% potassium diglyceryl monostearate citrate |
| Polar solubility | 4% water |
| promoter: | |

Potassium diglyceryl monostearate citrate is the potassium salt of the diester of citric acid and glyceryl monostearate.

Emulsifier 3:

| | |
|---|---|
| Emulsifier component A: | 85% sorbitan laurate¹⁾ |
| | 8% polyglyceryl-4 laurate²⁾ |
| Emulsifier component B: | 3% potassium dilauryl citrate |
| Polar solubility promoter: | 4% water |

Potassium dilauryl citrate is the potassium salt of the diester of citric acid and lauryl alcohol.

Emulsifier 4:

| | |
|---|---|
| Emulsifier component A: | 80% sorbitan laurate¹⁾ |
| | 14% polyglyceryl-10 laurate |
| Emulsifier component B: | 2% potassium diglyceryl monolaurate citrate |
| Polar solubility | 4% water |
| promoter: | |

Comparison emulsifier 2:
Exclusively emulsifier component A (to 100%; i.e., 85% sorbitan laurate and 15% polyglyceryl-10 laurate)

Emulsifier 5:

| | |
|---|---|
| Emulsifier component A: | 95% sorbitan laurate¹⁾ |
| Emulsifier component B: | 3% potassium diglyceryl monolaurate citrate |
| Polar solubility | 2% water |
| promoter: | |

Comparison emulsifier 3:
Exclusively emulsifier component A (i.e. 100% of sorbitan laurate)

Emulsifier 6:

| | |
|---|---|
| Emulsifier component A: | 98% polyglyceryl-4 laurate²⁾ |
| Emulsifier component B: | 0.3% potassium diglyceryl monolaurate citrate |
| | 0.2% sodium lauryl sulfate |
| Polar solubility | 1.5% water |
| promoter: | |

Emulsifier 7:

| | |
|---|---|
| Emulsifier component A: | 80% sorbitan laurate¹⁾ |
| | 12% polyglyceryl-4 laurate²⁾ |
| Emulsifier component B: | 3% potassium lauryl sulfate |
| Polar solubility promoter: | 5% water |

Emulsifier 8:

| | |
|---|---|
| Emulsifier component A: | 64% sorbitan oleate³⁾ |
| | 28% polyglyceryl-4 caprate⁴⁾ |
| Emulsifier component B: | 3% potassium diglyceryl monostearate |
| | citrate |
| Polar solubility promoter: | 5% water |

³⁾TEGO ® SMO V (Degussa);
⁴⁾TEGOSOFT ® PC 41 (Degussa)

| | | | | | | | C1 | C2 |
|---|---|---|---|---|---|---|---|---|
| | | 1 | 2 | 3 | 4 | 5 | (cf. 1, 3, 5) | (cf. 2, 4) |
| A | Emulsifier 1 | 1.50% | 1.50% | | | | | |
| | Comparison | | | | | | 1.50% | 1.50% |
| | emulsifier 1 | | | | | | | |
| | Emulsifier 2 | | | 1.50% | 1.50% | | | |
| | Emulsifier 3 | | | | | 1.50% | | |
| | Capryl/capric | | 18.50% | | 18.50% | | | 18.50% |
| | triglycerides | | | | | | | |
| | Paraffinum | 18.50% | | 18.50% | | 18.50% | 18.50% | |
| | perliquidum | | | | | | | |
| | Ethylhexyl | 1.10% | 1.10% | 1.10% | 1.10% | 1.10% | 1.10% | 1.10% |
| | palmitate | | | | | | | |
| | Xanthan gum | 0.20% | 0.20% | 0.20% | 0.20% | 0.20% | 0.20% | 0.20% |
| | Carbomer | 0.20% | 0.20% | 0.20% | 0.20% | 0.20% | 0.20% | 0.20% |
| B | Glycerol | 2.35% | 2.35% | 2.35% | 2.35% | 2.35% | 2.35% | 2.35% |
| | Water | ad 100% | ad 100% | ad 100% | ad 100% | ad 100% | ad 100% | ad 100% |
| C | NaOH (10%) | q.s. | q.s. | q.s. | q.s. | q.s. | q.s. | q.s. |
| | Preservative, | q.s. | q.s. | q.s. | q.s. | q.s. | q.s. | q.s. |
| | perfume | | | | | | | |
| | Stability | very | very | very | very | very | unstable | unstable |
| | | good | good | good | good | good | | |
| | | | | | | C3 | C4 | C2 |
| | | 6 | 7 | 8 | 9 | (cf. 6) | (cf. 7) | (cf. 8) |
| A | Emulsifier 4 | 1.50% | 1.50% | | | | | |
| | Emulsifier 5 | | | 1.50% | | | | |
| | Emulsifier 6 | | | | 1.50% | | | |
| | Comparison | | | | | 1.50% | 1.50% | |
| | emulsifier 2 | | | | | | | |
| | Comparison | | | | | | | 1.50% |
| | emulsifier 3 | | | | | | | |
| | Capryl/capric | | 18.50% | | 18.50% | | 18.50% | |
| | triglycerides | | | | | | | |
| | Paraffinum | 18.50% | | 18.50% | | 18.50% | | 1.850% |
| | perliquidum | | | | | | | |
| | Ethylhexyl | 1.10% | 1.10% | 1.10% | 1.10% | 1.10% | 1.10% | 1.10% |
| | palmitate | | | | | | | |
| | Xanthan gum | 0.20% | 0.20% | 0.20% | 0.20% | 0.20% | 0.20% | 0.20% |
| | Carbomer | 0.20% | 0.20% | 0.20% | 0.20% | 0.20% | 0.20% | 0.20% |
| B | Glycerol | 2.35% | 2.35% | 2.35% | 2.35% | 2.35% | 2.35% | 2.35% |
| | Water | ad 100% | ad 100% | ad 100% | ad 100% | ad 100% | ad 100% | ad 100% |
| C | NaOH (10%) | q.s. | q.s. | q.s. | q.s. | q.s. | q.s. | q.s. |
| | Preservative, | q.s. | q.s. | q.s. | q.s. | q.s. | q.s. | q.s. |
| | perfume | | | | | | | |
| | Stability | very | very | good | very | unstable | unstable | unstable |
| | | good | good | | good | | | |

| | | | | | | | | C6 |
|---|---|---|---|---|---|---|---|---|
| | | 10 | 11 | 12 | 13 | 14 | 15 | (cf. 10, 11, 12) |
| A | Emulsifier 1 | 1.50% | | | | | | |
| | Emulsifier 2 | | 1.50% | | | | | |
| | Emulsifier 3 | | | 1.50% | | | | |
| | Comparison | | | | | | | 1.50% |
| | emulsifier 1 | | | | | | | |
| | Emulsifier 7 | | | | 1.50% | | | |
| | Emulsifier 8 | | | | | 1.50% | 1.50% | |
| | Capryl/capric | | | | | | 18.50% | |
| | triglycerides | | | | | | | |
| | Paraffinum | | | | | 18.50% | | |
| | perliquidum | | | | | | | |
| | Ethylhexyl | 19.60% | 19.60% | 19.60% | 19.60% | 1.10% | 1.10% | 19.60% |
| | palmitate | | | | | | | |
| | Xanthan gum | 0.20% | 0.20% | 0.20% | 0.20% | 0.20% | 0.20% | 0.20% |
| | Carbomer | 0.20% | 0.20% | 0.20% | 0.20% | 0.20% | 0.20% | 0.20% |
| B | Glycerol | 2.35% | 2.35% | 2.35% | 2.35% | 2.35% | 2.35% | 2.35% |
| | Water | ad 100% | ad 100% | ad 100% | ad 100% | ad 100% | ad 100% | ad 100% |
| C | NaOH (10%) | q.s. | q.s. | q.s. | q.s. | q.s. | q.s. | q.s. |
| | Preservative, | q.s. | q.s. | q.s. | q.s. | q.s. | q.s. | q.s. |
| | perfume | | | | | | | |
| | Stability | very | very | very | very | good | good | unstable |
| | | good | good | good | good | | | |

While the present invention has been particularly shown and described with respect to preferred embodiments thereof, it will be understood by those skilled in the art that the foregoing and other changes in forms and details may be made without departing from the spirit and scope of the present invention. It is therefore intended that the present invention not be limited to the exact forms and details described and illustrated, but fall within the scope of the appended claims.

What is claimed is:

1. A liquid, cold-processable oil-in-water emulsifier system comprising a liquid, PEG-free emulsifier base consisting of
A) one or more polyol partial esters; and
Ba) one or more acid partial esters carrying neutralizable acid functions, or
Bb) one or more acid partial esters carrying at least partially neutralized acid functions.

2. The liquid, cold-processable oil-in-water emulsifier system of claim 1 wherein said liquid, PEG-free emulsifier base further includes at least one polar liquid solubility promoter.

3. The liquid, cold-processable oil-in-water emulsifier system as claimed in claim 2, which is a single phase.

4. The liquid, cold-processable oil-in-water emulsifier system as claimed in claim 1, which comprises, as liquid base component (A) one or more emulsifiers selected from at least one of the following groups:
A1) sorbitan or sorbitol partial esters,
A2) glycerol and polyglycerol partial esters,
A3) carbohydrate esters, and
A4) (alkyl poly)glycosides.

5. The liquid, cold-processable oil-in-water emulsifier system as claimed in claim 1, which comprises, as emulsifier component (B), one or more emulsifiers selected from at least one of the following groups:
B1) Di- or polycarboxylates optionally containing hydroxyl groups, sulfated or sulfonated carboxylates, malonates, malates, succinates, sulfosuccinates, citrates, tartrates in which the acid groups have been partially esterified with aliphatic or aromatic, linear or branched, optionally unsaturated and/or hydroxy-functionalized alcohols with a chain length of from 6 to 22 carbon atoms,
B2) Di- or polycarboxylates optionally containing hydroxyl groups, sulfated or sulfonated or phosphated carboxylates, malonates, malates, succinates, sulfosuccinates, citrates, tartrates in which the acid groups have been partially esterified with polyols, polyol partial esters, with aliphatic or aromatic, linear or branched, optionally unsaturated and/or hydroxy-functionalized carboxylic acids with a chain length of from 6 to 22 carbon atoms,
B3) polyols, which are partially esterified with aliphatic or aromatic, linear or branched, optionally unsaturated and/or hydroxy-functionalized mono-, di- or polycarboxylic acids with a chain length of from 2 to 22 carbon atoms, with the proviso that there are free, neutralizable acid groups in the molecule,
B4) hydroxy-functional, mono-, di or polycarboxylic acids whose hydroxyl groups have been reacted at least partially with aliphatic, linear or branched, optionally unsaturated and/or hydroxy-functionalized carboxylic acids with a chain length of from 6 to 22 carbon atoms,
B5) N-acylamino acids comprising an aliphatic or aromatic, linear or branched, optionally unsaturated and/or hydroxy-functionalized radical with a chain length of from 6 to 22 carbon atoms, and
B6) carboxylates, sulfates, sulfonates or phosphates, comprising an aliphatic or aromatic, linear or branched, optionally unsaturated and/or hydroxy functionalized radical with a chain length of from 6 to 22 carbon atoms.

6. The liquid, cold-processable oil-in-water emulsifier system as claimed in claim 1, which comprises sorbitan esters and polyglycerol esters as liquid emulsifier component (A).

7. The liquid, cold-processable oil-in-water emulsifier system as claimed in claim 6, which comprises, as liquid emulsifier component (A), sorbitan laurate and polyglycerol laurate.

8. The liquid, cold-processable oil-in-water emulsifier system as claimed in claim 1, which comprises, as emulsifier component, (Ba) citric acid partial esters carrying neutralizable acid functions, or (Bb) citric acid partial esters carrying acid functions, at least partially neutralized.

9. The liquid, cold-processable oil-in-water emulsifier system as claimed in claim 1, which comprises the emulsifier components (B) in amounts of at most 20% by weight, based on the emulsifier base (A).

10. The liquid, cold-processable oil-in-water emulsifier system as claimed in claim 1, wherein the hydrophobic ester groups of the emulsifier systems (A) and/or (B) are lauric acid or lauric alcohol radicals.

11. The liquid, cold-processable oil-in-water emulsifier systems as claimed in claim 2, wherein the liquid polar solubility promoter (C) is at least one compound selected from water, glycols, polyalkylene glycols, glycerols, polyglycerols, and alcohols.

12. An oil-in-water emulsion comprising an emulsifier system of
A) one or more polyol partial esters; and
Ba) one or more acid partial esters carrying neutralizable acid functions, or
Bb) one or more acid partial esters carrying at least partially neutralized acid functions.

13. A preparation comprising the emulsifying system of claim 1.
